(19) United States
(12) Patent Application Publication
(10) Pub. No.: US 2024/0009107 A1
(43) Pub. Date: Jan. 11, 2024

(54) HYALURONIC ACID COMPOSITION HAVING PERMEATION-PROMOTING EFFECT, PREPARATION METHOD THEREFOR AND USE THEREOF

(71) Applicants: BLOOMAGE BIOTECH (TIANJIN) CO., LTD., Tianjin (CN); BLOOMAGE BIOTECHNOLOGY CORPORATION LIMITED, Shandong (CN)

(72) Inventors: Yuling WANG, Shandong (CN); Xueping GUO, Shandong (CN); Huixia LV, Shandong (CN); Liping QIAO, Shandong (CN); Shujing REN, Shandong (CN); Ximing WEN, Shandong (CN); Xia LI, Shandong (CN); Jing WANG, Shandong (CN); Feng GENG, Shandong (CN)

(73) Assignees: BLOOMAGE BIOTECH (TIANJIN) CO., LTD., Tianjin (CN); BLOOMAGE BIOTECHNOLOGY CORPORATION LIMITED, Shandong (CN)

(21) Appl. No.: 18/036,471

(22) PCT Filed: Nov. 5, 2021
(86) PCT No.: PCT/CN2021/128882

§ 371 Date: May 11, 2023

(30) Foreign Application Priority Data
Nov. 11, 2020 (CN) 202011256392.X

(51) Int. Cl. A61K 8/73; A61Q 19/00; A61K 31/195; A61K 31/122; A61K 47/36; A61K 8/44; A61K 8/35; A61L 26/00

(52) U.S. Cl. CPC A61K 8/735; A61Q 19/00; A61K 31/195; A61K 31/122; A61K 47/36; A61K 8/44; A61K 8/35; A61L 26/0023; A61L 26/0066; A61K 2800/10; A61L 2300/214; A61L 2300/216

(57) ABSTRACT

Disclosed are a hyaluronic acid composition having permeation-promoting effects, a preparation method thereof and the use thereof. Provided is a hyaluronic acid composition, comprising: hyaluronic acid or a salt thereof, an acetylated hyaluronic acid or a salt thereof, and a hydrolyzed hyaluronic acid or a salt thereof. The hyaluronic acid composition of the present application has a small addition amount, can effectively facilitate the absorption of other active ingredients in a formula to achieve synergistic effects, has a small particle size, is uniform, has a fast dissolution rate, has an excellent absorption speed in a product, and is superior to hyaluronic acid treated by means of simple mixing in improving skin hydration. Compared with traditional penetration promoters, the hyaluronic acid composition of the present application causes no damage to the skin and is safe.

### TECHNICAL FIELD

This application relates to the technical field of hyaluronic acid, in particular to a hyaluronic acid composition with penetration-promoting effect, a preparation method and use thereof.

### BACKGROUND ART

From ancient times to the present, the love of beauty is the eternal pursuit of people, especially women. With the growth of age, the skin appears dull, dry, loose, sagging, and wrinkles and so on, which bothers all people. Therefore, most young people realized that in order to maintain the skin's youthful characteristics such as moisture, whiteness, elasticity, and water-oil balance, skin care should be carried out as early as possible to delay the aging speed of skin.

Skin is the largest organ of human body. The average surface area of skin of an adult is about 1.6 m², and the total weight accounts for about 16% of the body weight. The skin is divided into three parts: the epidermis, the dermis and the subcutaneous tissue from the outside to the inside. Skin is not only a decorative coat for humans, but also has a strong protective effect. The brick wall structure of the epidermis allows the skin to block foreign substances from the skin, thereby protecting our human body. On the other hand, if the skin wants to achieve rejuvenation, the active substances in skin care products must enter the target layer of the skin to play a role. Therefore, in the field of skin care products, it is particularly important to promote the absorption of nutrients.

In the field of drug development, formulators often use propylene glycol, azone, menthol, borneol, etc. as penetration enhancers to promote the absorption of drugs and exert local therapeutic effects. Because drugs are only used when sick, and generally used topically, adverse reactions such as sensitivity, redness, etc. will not be considered more carefully. Cosmetics are preparations that are used every morning and evening, so it is particularly necessary for more skin-safe penetration-enhancing ingredients.

MB Brown (European Academy of Dermatology and Venereology, 2005: 19, 308-318) and so on compared sodium hyaluronate as a drug matrix with water, chondroitin sulfate, and sodium carboxymethylcellulose as a matrix, and found that HA can significantly strengthen the distribution of diclofenac sodium in all layers of skin, and retain and locate the drug in the epidermal layer. The concentration of HA in this experiment was 2.5%.

In CN202010407830.1, a patent application for a composition containing hyaluronic acid and polyols and application thereof, the inventor disclosed a composition of hyaluronic acid and polyols, which can increase the transdermal absorption of water-soluble amino acid components, so that it can enter the epidermis and dermis without the aid of equipment, provide effects and exert cosmetic effects. The composition is composed of high, low and oligomeric hyaluronic acids or salts thereof, and polyol antioxidants, with relatively complex components.

However, at present, there is no report on the promotion of target actives by purely using a hyaluronic acid composition.

### BRIEF SUMMARY

Based on the actual needs of current skin care products development and the problems existing in the current penetration enhancing system, the present application combines a hydrolyzed hyaluronic acid or a salt thereof, a hyaluronic acid or a salt thereof and an acetylated hyaluronic acid or a salt thereof. When applied to the formula, the transdermal absorption rate of the target actives can be significantly improved.

The specific technical solutions of the present application are as follows:

1. A hyaluronic acid composition comprising:
a hyaluronic acid or a salt thereof;
an acetylated hyaluronic acid or a salt thereof; and
a hydrolyzed hyaluronic acid or a salt thereof.

2. The hyaluronic acid composition according to item 1, wherein,
the hyaluronic acid composition consists of a hyaluronic acid or a salt thereof, an acetylated hyaluronic acid or a salt thereof, and a hydrolyzed hyaluronic acid or a salt thereof.

3. The hyaluronic acid composition according to item 1 or 2, wherein based on the weight percentage in the hyaluronic acid composition,
the hyaluronic acid or a salt thereof is 20-60%, preferably 25-55%, more preferably 2540%;
the acetylated hyaluronic acid or a salt thereof is 10-50%, preferably 1545%, more preferably 20-35%; and
the hydrolyzed hyaluronic acid or a salt thereof is 30-70%, preferably 30-60%, more preferably 40-55%.

4. The hyaluronic acid composition according to any one of items 1-3, wherein, the molecular weight of the hyaluronic acid or a salt thereof is 100 k-500 kDa, preferably 150 k-300 kDa, more preferably 210 k-300 kDa;
the molecular weight of the acetylated hyaluronic acid or a salt thereof is 10 k-100 kDa, preferably 10 k-50 kDa, more preferably 20 k-30 kDa; and the molecular weight of the hydrolyzed hyaluronic acid or a salt thereof is 0.8 k-20 kDa, preferably 3 k-15 kDa, more preferably 3 k-10 kDa.

5. The composition according to any one of items 14, wherein the acetyl content of the acetylated hyaluronic acid or a salt thereof is 20-30 wt %.

6. A method for preparing a hyaluronic acid composition, comprising the following steps of:
dissolving a hyaluronic acid or a salt thereof, an acetylated hyaluronic acid or a salt thereof and a hydrolyzed hyaluronic acid or a salt thereof to obtain a solution; spray-drying the resulting solution to obtain a hyaluronic acid composition.

7. The method according to item 6, wherein based on the mass percentage in the solution, the sum of the hyaluronic acid or a salt thereof, acetylated hyaluronic acid or a salt thereof and hydrolyzed hyaluronic acid or a salt thereof is 1-10%.

8. The method according to item 6 or 7, wherein the feeding temperature of spray-drying is 120-150° C., the discharging temperature is 80-100° C., preferably, the atomization frequency is 30-50 Hz.

9. The method according to any one of items 6-8, wherein based on the weight percentage in the hyaluronic acid composition,
the hyaluronic acid or a salt thereof is 20-60%, preferably 25-55%, more preferably 25-40%;
the acetylated hyaluronic acid or a salt thereof is 10-50%, preferably 15-45%, more preferably 20-35%; and
the hydrolyzed hyaluronic acid or a salt thereof is 30-70%, preferably 30-60%, more preferably 40-55%.

10. The method according to any one of items 6-9, wherein
the molecular weight of the hyaluronic acid or a salt thereof is 100 k-500 kDa, preferably 150 k-300 kDa, more preferably 210 k-300 kDa;
the molecular weight of the acetylated hyaluronic acid or a salt thereof is 10 k-100 kDa, preferably 10 k-50 kDa, more preferably 20 k-30 kDa; and
the molecular weight of the hydrolyzed hyaluronic acid or a salt thereof is 0.8 k-20 kDa, preferably 3 k-15 kDa, more preferably 3 k-10 kDa.

11. The method according to any one of items 6-10, wherein the acetyl content of the acetylated hyaluronic acid or a salt thereof is 20%-30%.

12. A composition for promoting the absorption of active ingredients, wherein the composition comprises an active ingredient and the hyaluronic acid composition according to any one of items 1-5 or the hyaluronic acid composition prepared by the preparation method according to any one of items 6-11.

13. The composition according to item 12, wherein the active ingredient is a water-soluble active ingredient and/or an oil-soluble active ingredient; preferably, the water-soluble active ingredient is tranexanuc acid, nicotinamide, vitamin C, ergothioneine, small molecule peptides containing 2-10 amino acids, aminobutyric acid, deoxyribonucleic acid, pro-xylane or ectoine; and the oil-soluble active ingredient is astaxanthin, salicylic acid, ferulic acid, phenylethyl resorcinol, resveratrol, undecylenoyl phenylalanine or ethyl bis(iminomethyl)guaiacol manganese chloride.

14. The composition according to item 12 or 13, wherein the mass ratio of the active ingredient to the hyaluronic acid composition is 1:5 to 5:1, preferably 1:4 to 4:1.

15. Use of the hyaluronic acid composition according to any one of items 1-5 or the hyaluronic acid composition prepared by the preparation method described in any one of items 6-11 in improving the absorption of active ingredient, preferably, the active ingredient is a water-soluble active ingredient and/or an oil-soluble active ingredient.

16. Use according to item 15, wherein the water-soluble active ingredient is tranexamic acid, nicotinamide, vitamin C, ergothioneine, small molecule peptides containing 2-10 amino acids, aminobutyric acid, deoxyribonucleic acid, pro-xylane or ectoine; and the oil-soluble active ingredient is astaxanthin, salicylic acid, ferulic acid, phenylethyl resorcinol, resveratrol, undecylenoyl phenylalanine, or ethyl bis(iminomethyl)guaiacol manganese chloride.

17. A product, comprising the composition according to any one of items 12-14.

18. The product according to item 17, wherein, based on the mass percentage in the product, the hyaluronic acid composition is 0.1-2%, preferably 0.5-1.5%.

19. The product according to item 17 or 18, wherein the product is a skin care product, a disinfection product, a medicine or a dressing, or a gel medical device.

20. The product according to item 19, wherein the skin care product is toner, essence, cream, mask or lotion.

21. Use of the hyaluronic acid composition according to any one of items 1-5 or the hyaluronic acid composition prepared by the preparation method according to any one of items 6-11 in the field of products, preferably, the hyaluronic acid composition is 0.1-2%, preferably 0.5-1.5%, based on the weight percentage in the product.

22. The use according to item 21, wherein the product is a skin care product, a disinfection product, a medicine or a dressing, or a gel medical device.

### Effects of the Application

1) The composition of hyaluronic acid or a salt thereof with penetration-promoting effect of the present application has a small amount of addition, which can effectively promote the absorption of other active ingredients in the formula and achieve a synergistic effect.

2) The composition of hyaluronic acid or a salt thereof with penetration-promoting effect of the present application has small particle size, uniformity, fast dissolution rate, excellent absorption rate in the product, and is superior to hyaluronic acid processed by simple mixing in improving skin hydration degree.

3) Compared with traditional penetration enhancers, the combination of hyaluronic acid or a salt thereof with penetration enhancing effect of the present application has no damage to the skin and is safe.

### DESCRIPTION OF DRAWINGS

FIG. 1 is the linear equation of tranexamic acid in Application Verification Example 1.

FIG. 2 is a schematic diagram of the cumulative permeation amount per unit area of tranexamic acid in the in vitro permeation test using the hyaluronic acid composition obtained in Example 6 and the reference product in Application Verification Example 1.

FIG. 3 is a schematic diagram of the skin storage amount of tranexamic acid at different times during the experiment using the hyaluronic acid composition obtained in Example 6 in Application Verification Example 1.

FIG. 4 is a linear equation of astaxanthin in Application Verification Example 2.

### DETAILED DESCRIPTION

The present application is described in detail below with reference to the embodiments described in the drawings, wherein the same numbers in all the drawings represent the same features. Although specific embodiments of the application are shown in the drawings, it should be understood that the application may be embodied in various forms and should not be limited by the embodiments set forth herein. Rather, these embodiments are provided for more thorough understanding of the present application and fully conveying the scope of the present application to those skilled in the art.

It should be noted that certain terms are used in the specification and claims to refer to specific components. Those skilled in the art should understand that they may use different nouns to refer to the same component. The specification and claims do not use differences in nouns as a way of distinguishing components, but use differences in functions of components as a criterion for distinguishing. “Comprising” or “including” mentioned throughout the specification and claims are open-ended terms, so it should be interpreted as “including but not limited to”. The following descriptions in the specification are preferred embodiments. However, the descriptions are for the purpose of the general principles of the specification, and are not intended to limit the scope of the present application. The protection scope of the present application should be defined by the appended claims.

The present application provides a hyaluronic acid composition, which comprises a hyaluronic acid or a salt thereof, an acetylated hyaluronic acid or a salt thereof and a hydrolyzed hyaluronic acid or a salt thereof.

The acetylated hyaluronic acid or a salt thereof is obtained by acetylation of hyaluronic acid or a salt thereof, and the introduction of acetyl group makes the acetylated hyaluronic acid or a salt thereof lipophilic.

The hydrolyzed hyaluronic acid or a salt thereof is oligomeric hyaluronic acid or a salt thereof produced by enzymatic degradation technology, which has smaller molecular weight, and is more easily transdermally absorbed into the epidermis and dermis.

In a preferred specific embodiment of the present application, wherein, based on the weight percentage in the hyaluronic acid composition, the hyaluronic acid or a salt thereof is 20-60%, preferably 25-55%, more preferably 25-40%; the acetylated hyaluronic acid or a salt thereof is 10-50%, preferably 15-45%, more preferably 20-35%; and the hydrolyzed hyaluronic acid or a salt thereof is 30-70%, preferably 30-60%, more preferably 40-55%.

For example, based on the weight percentage in the hyaluronic acid composition, the content of the hyaluronic acid or a salt thereof can be 20%, 25%, 30%, 35%, 40%, 45%, 50%, 55%, 60% or any range in between;
the acetylated hyaluronic acid or a salt thereof can be 10%, 15%, 20%, 25%, 30%, 35%, 40%, 45%, 50% or any range in between;
the hydrolyzed hyaluronic acid or a salt thereof can be 30%, 35%, 40%, 45%, 50%, 55%, 60%, 65%, 70% or any range in between.

In a preferred specific embodiment of the present application, wherein, the molecular weight of the hyaluronic acid or a salt thereof is 100 k-500 kDa, preferably 150 k-300 kDa, more preferably 210 k-300 kDa; the molecular weight of the acetylated hyaluronic acid or a salt thereof is 10 k-100 kDa, preferably 10 k-50 kDa, more preferably 20 k-30 kDa; and the molecular weight of the hydrolyzed hyaluronic acid or a salt thereof is 0.8 k-20 kDa, preferably 3 k-15 kDa, more preferably 3 k-10 kDa.

For example, the molecular weight of the hyaluronic acid or a salt thereof can be 100 kDa, 110 kDa, 120 kDa, 130 kDa, 140 kDa, 150 kDa, 160 kDa, 170 kDa, 180 kDa, 190 kDa, 200 kDa, 210 kDa, 220 kDa, 230 kDa, 240 kDa, 250 kDa, 260 kDa, 270 kDa, 280 kDa, 290 kDa, 300 kDa, 350 kDa, 400 kDa, 450 kDa, 500 kDa or any range in between;
the molecular weight of the acetylated hyaluronic acid or a salt thereof can be 10 kDa, 20 kDa, 30 kDa, 40 kDa, 50 kDa, 60 kDa, 70 kDa, 80 kDa, 90 kDa, 100 kDa or any range in between;
the molecular weight of the hydrolyzed hyaluronic acid or a salt thereof can be 0.8 kDa, 0.9 kDa, 1 kDa, 2 kDa, 3 kDa, 4 kDa, 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa or any range in between.

In a preferred specific embodiment of the present application, wherein the acetyl content of the acetylated hyaluronic acid or a salt thereof is 20-30 wt %.

For example, the acetyl content of acetylated hyaluronic acid or a salt thereof can be 20 wt %, 21 wt %, 22 wt %, 23 wt %, 24 wt %, 25 wt %, 26 wt %, 27 wt %, 28 wt %, 29 wt %, 30 wt % or any range in between.

In a preferred specific embodiment of the present application, wherein the hyaluronic acid composition consists of hyaluronic acid or a salt thereof, acetylated hyaluronic acid or a salt thereof, and hydrolyzed hyaluronic acid or a salt thereof.

The hyaluronic acid salt, acetylated hyaluronic acid salt or hydrolyzed hyaluronic acid salt refers to a metal ion salt, such as a sodium salt, a potassium salt, a calcium salt, a zinc salt, etc., and the sodium salt is commonly used.

In a preferred specific embodiment of the present application, wherein the hyaluronic acid composition comprises a hyaluronic acid or a salt thereof; an acetylated hyaluronic acid or a salt thereof; and a hydrolyzed hyaluronic acid or a salt thereof:
based on the weight percentage in the hyaluronic acid composition,
the hyaluronic acid or a salt thereof is 20-60%, preferably 25-55%, more preferably 25-40%;
the acetylated hyaluronic acid or a salt thereof is 10-50%, preferably 15-45%, more preferably 20-35%; and
the hydrolyzed hyaluronic acid or a salt thereof is 30-70%, preferably 30-60%, more preferably 40-55%.

In a preferred specific embodiment of the present application, wherein the hyaluronic acid composition comprises a hyaluronic acid or a salt thereof; an acetylated hyaluronic acid or a salt thereof; and a hydrolyzed hyaluronic acid or a salt thereof:
based on the weight percentage in the hyaluronic acid composition,
the hyaluronic acid or a salt thereof is 20-60%, preferably 25-55%, more preferably 25-40%;
the acetylated hyaluronic acid or a salt thereof is 10-50%, preferably 15-45%, more preferably 20-35%; and
the hydrolyzed hyaluronic acid or a salt thereof is 30-70%, preferably 30-60%, more preferably 40-55%;
the molecular weight of the hyaluronic acid or a salt thereof is 100 k-500 kDa, preferably 150 k-300 kDa, more preferably 210 k-300 kDa;
the molecular weight of the acetylated hyaluronic acid or a salt thereof is 10 k-100 kDa, preferably 10 k-50 kDa, more preferably 20 k-30 kDa; and
the molecular weight of the hydrolyzed hyaluronic acid or a salt thereof is 0.8 k-20 kDa, preferably 3 k-15 kDa, more preferably 3 k-10 kDa.

In a preferred specific embodiment of the present application, wherein the hyaluronic acid composition comprises a hyaluronic acid or a salt thereof; an acetylated hyaluronic acid or a salt thereof; and a hydrolyzed hyaluronic acid or a salt thereof;
based on the weight percentage in the hyaluronic acid composition,
the hyaluronic acid or a salt thereof is 20-60%, preferably 25-55%, more preferably 25-40%;
the acetylated hyaluronic acid or a salt thereof is 10-50%, preferably 1545%, more preferably 20-35%; and
the hydrolyzed hyaluronic acid or a salt thereof is 30-70%, preferably 30-60%, more preferably 40-55%;
the molecular weight of the hyaluronic acid or a salt thereof is 100 k-500 kDa, preferably 150 k-300 kDa, more preferably 210 k-300 kDa;
the molecular weight of the acetylated hyaluronic acid or a salt thereof is 10 k-100 kDa, preferably 10 k-50 kDa, more preferably 20 k-30 kDa; and
the molecular weight of the hydrolyzed hyaluronic acid or a salt thereof is 0.8 k-20 kDa, preferably 3 k-15 kDa, more preferably 3 k-10 kDa, and the acetyl content of the acetylated hyaluronic acid or a salt thereof is 20-30 wt %.

The hyaluronic acid composition provided by the present application is presumed to have the following mechanism of action: a hyaluronic acid or a salt thereof is equivalent to a very small network structure, which is distributed on the surface of the skin; an acetylated hyaluronic acid or a salt thereof has acetyl and is lipophilic, thus it can be skin friendly with the skin and form channels; a hydrolyzed hyaluronic acid or a salt thereof can quickly penetrate into the skin layer through the channels formed by the acetylated hyaluronic acid or a salt thereof, the hydrolyzed hyaluronic acid or a salt thereof can quickly open the skin barrier by mixing with the hyaluronic acid or a salt thereof, and nutrients, the hydrolyzed hyaluronic acid or a salt thereof, and nutrients combined with the hyaluronic acid or a salt thereof can enter the skin, promoting the absorption of active molecules, such as promoting the absorption of oil-soluble active ingredients or water-soluble active ingredients in skin care products, and promoting the absorption of other active ingredients in disinfection products, medicines, dressings or gel medical devices.

The present application provides a method for preparing hyaluronic acid composition, comprising the following steps:
dissolving a hyaluronic acid or a salt thereof, an acetylated hyaluronic acid or a salt thereof and a hydrolyzed hyaluronic acid or a salt thereof to obtain a solution;
spray-drying the resulting solution to obtain a hyaluronic acid composition.

In a preferred specific embodiment of the present application, wherein the feeding temperature of spray-drying is 120-150° C., the discharging temperature is 80-100° C., preferably, the atomization frequency is 30-50 Hz.

For example, the feeding temperature of spray-drying can be 120° C., 125° C., 130° C., 135° C., 140° C., 145° C., 150° C. or any range in between:
the discharging temperature can be, for example, 80° C., 85° C., 90° C., 95° C., 100° C. or any range in between.

The atomization frequency can be, for example, 30 Hz, 35 Hz, 40 Hz, 45 Hz, 50 Hz or any range in between.

In a preferred specific embodiment of the present application, wherein the hyaluronic acid composition obtained after the spray-drying treatment is fine particles that can pass through 100-200 mesh.

The present application provides a composition for promoting absorption of active ingredients, wherein the composition comprises an active ingredient and the above-mentioned hyaluronic acid composition or the hyaluronic acid composition prepared by the above-mentioned preparation method.

The active ingredient can be an active ingredient known to those skilled in the art, such as active ingredients in the field of skin care products, active ingredients in medicines, active ingredients in disinfection products, active ingredients in dressings, or active ingredients in gel medical devices.

The gel medical devices refer to medical devices such as oral and nasal cavity sprays and repair gels.

In a preferred specific embodiment of the present application, wherein the active ingredients are water-soluble active ingredients and/or oil-soluble active ingredients.

The water-soluble active ingredients refer to active ingredients that can be dissolved in water, and they can be any water-soluble active ingredient known to those skilled in the art that can be used in skin care products or gel medical devices, such as tranexamic acid, niacinamide, vitamin C, ergothioneine, small molecular peptides containing 2-10 amino acids, aminobutyric acid, deoxyribonucleic acid, pro-xylane, or ectoine.

The oil-soluble active ingredients refer to active ingredients that are insoluble in water, and they can be any oil-soluble active ingredient that can be used in skin care products or gel medical devices known to those skilled in the art, such as astaxanthin, salicylic acid, ferulic acid, phenylethyl resorcinol, resveratrol, undecylenoyl phenylalanine, or ethyl bis(imino methyl)guaiacol manganese chloride.

In a preferred specific embodiment of the present application, wherein, the mass ratio of the active ingredient to the hyaluronic acid composition is 1:5 to 5:1, preferably 1:4 to 4:1.

For example, the mass ratio of the active ingredient to the hyaluronic acid composition (active ingredient/hyaluronic acid composition) is 1:5, 1:4, 1:3, 1:2, 1:1, 2:1, 3:1, 4:1, 5:1 or any range in between.

The present application provides the use of the above-mentioned hyaluronic acid composition or the hyaluronic acid composition prepared by the above-mentioned preparation method in improving the absorption of active ingredients; preferably, the active ingredients are water-soluble active ingredients and/or oil-soluble active ingredients.

In a preferred specific embodiment of the present application, wherein the water-soluble active ingredient is tranexamic acid, nicotinamide, vitamin C, ergothioneine, small molecule peptides containing 2-10 amino acids, aminobutyric acid, deoxyribonucleic acid, pro-xylane, or ectoine; and the oil-soluble active ingredient is astaxanthin, salicylic acid, ferulic acid, phenylethyl resorcinol, resveratrol, undecylenoyl phenylalanine, or ethyl bis(imino methyl)guaiacol manganese chloride.

The present application provides a product comprising the composition described above.

In a preferred specific embodiment of the present application, the hyaluronic acid composition is 0.1-2%, preferably 0.5-1.5%, based on the mass percentage in the product.

For example, the hyaluronic acid composition can be 0.1%, 0.2%, 0.3%, 0.4%, 0.5%, 0.6%, 0.7%, 0.8%, 0.9%, 1%, 2% or any range in between, based on the mass percentage in the product.

In a preferred specific embodiment of the present application, wherein the product is a skin care product, a disinfection product, a medicine or a dressing, or a gel medical device.

In a preferred specific embodiment of the present application, wherein the skin care product is toner, essence, cream, mask, lotion or other body care products.

In a preferred specific embodiment of the present application, wherein the product also comprises other auxiliary materials, the auxiliary materials are any known auxiliary materials in the art, for example, the auxiliary materials of the skin care products can be butanediol, pentanediol, glycerin, caprylic decanoate triglyceride, jojoba oil, hydrogenated lecithin, xanthan gum, carbomer, triethanolamine, poloxamer, tranexamic acid, astaxanthin, ethylparaben, etc.

The present application provides the use of the above-mentioned hyaluronic acid composition or the hyaluronic acid composition prepared by the above-mentioned preparation method in the field of products. Preferably, based on the weight percentage in the product, the hyaluronic acid composition is 0.1-2%, preferably 0.5-1.5%.

In a preferred specific embodiment of the present application, wherein the product is a skin care product, a disinfection product, a medicine or a dressing, or a gel medical device.

The present application uses the above-mentioned hyaluronic acid composition to promote the absorption of active ingredients in products such as skin care products, medicines, disinfection products, dressings or gel medical devices, thereby increasing the transdermal absorption rate of the active ingredients.

The present application generally and/or specifically describes the materials used in the test and test methods. In the following examples, if there is no other special description, % means wt %, i.e. weight percentage. The reagents or instruments used, whose manufacturers are not indicated, are all commercially available conventional reagent products.

TABLE 1
the source of raw materials used in the examples

| Name of the raw materials | purity | manufacturer |
|---|---|---|
| sodium hyaluronate | >92% | Bloomage Biotechnology |
| | | Corporation Limited |
| acetylated sodium hyaluronate | >92% | Bloomage Biotechnology |
| | | Corporation Limited |
| hydrolyzed sodium hyaluronate | >92% | Bloomage Biotechnology |
| | | Corporation Limited |
| tranexamic acid | >99% | commercially available |
| astaxanthin | >90% | commercially available |

### Example 1

Precisely weighed 20 g of sodium hyaluronate with molecular weight of 400 kDa, 10 g of acetylated sodium hyaluronate with molecular weight of 100 kDa, and 70 g of hydrolyzed sodium hyaluronate with molecular weight of 18 kDa. After mixing evenly, 1000 ml of purified water was added to make a 10% aqueous solution, which was then treated by spray-drying. Set the feeding temperature to 125° C., the discharging temperature to 90° C., and the atomizer frequency to 40 Hz to obtain uniform fine powder.

### Example 2

Precisely weighed 30 g of sodium hyaluronate with molecular weight of 250 kDa, 30 g of acetylated sodium hyaluronate with molecular weight of 30 kDa, and 40 g of hydrolyzed sodium hyaluronate with molecular weight of 10 kDa. After mixing evenly, 2000 ml of purified water was added to make a 5% aqueous solution, which was then treated by spray-drying. Set the feeding temperature to 125° C., the discharging temperature to 90° C. and the atomizer frequency to 40 Hz to obtain uniform fine powder.

### Example 3

Precisely weighed 35 g of sodium hyaluronate with molecular weight of 280 kDa, 25 g of acetylated sodium hyaluronate with molecular weight of 25 kDa, and 40 g of hydrolyzed sodium hyaluronate with molecular weight of 8 kDa. After mixing evenly, 3000 ml of purified water was added to make a 3.33% aqueous solution, which was then treated by spray-drying. Set the feeding temperature to 125° C., the discharging temperature to 90° C., and the atomizer frequency to 40 Hz to obtain uniform fine powder.

### Example 4

Precisely weighed 25 g of sodium hyaluronate with molecular weight of 300 kDa, 20 g of acetylated sodium hyaluronate with molecular weight of 30 kDa, and 55 g of hydrolyzed sodium hyaluronate with molecular weight of 5 kDa. After mixing evenly, 3000 ml of purified water was added to make a 3.33% aqueous solution, which was then treated by spray-drying. Set the feeding temperature to 125° C., the discharging temperature to 90° C., and the atomizer frequency to 40 Hz to obtain uniform fine powder.

### Example 5

Precisely weighed 40 g of sodium hyaluronate with molecular weight of 250 kDa, 35 g of acetylated sodium hyaluronate with molecular weight of 28 kDa, and 45 g of hydrolyzed sodium hyaluronate with molecular weight of 3 kDa. After mixing evenly, 3000 ml of purified water was added to make a 3.33% aqueous solution, which was then treated by spray-drying. Set the feeding temperature to 125° C., the discharging temperature to 90° C., and the atomizer frequency to 40 Hz to obtain uniform fine powder.

### Example 6

Precisely weighed 30 g of sodium hyaluronate with molecular weight of 210 kDa, 20 g of acetylated sodium hyaluronate with molecular weight of 20 kDa, and 50 g of hydrolyzed sodium hyaluronate with molecular weight of 3 kDa. After mixing evenly, 3000 ml of purified water was added to make a 3.33% aqueous solution, which was then treated by spray-drying. Set the feeding temperature to 125° C., the discharging temperature to 90° C., and the atomizer frequency to 40 Hz to obtain uniform fine powder.

### Example 7

Precisely weighed 50 g of sodium hyaluronate with molecular weight of 150 kDa, 15 g of acetylated sodium hyaluronate with molecular weight of 45 kDa, and 35 g of hydrolyzed sodium hyaluronate with molecular weight of 3 kDa. After mixing evenly, 1000 ml of purified water was added to make a 10% aqueous solution, which was then treated by spray-drying. Set the feeding temperature to 120° C., the discharging temperature to 100° C., and the atomizer frequency to 50 Hz to obtain uniform fine powder.

### Example 8

Precisely weighed 55 g of sodium hyaluronate with molecular weight of 300 kDa, 15 g of acetylated sodium hyaluronate with molecular weight of 10 kDa, and 30 g of hydrolyzed sodium hyaluronate with molecular weight of 15 kDa. After mixing evenly, 2000 ml of purified water was added to make a 5% aqueous solution, which was then treated by spray-drying. Set the feeding temperature to 140° C., the discharging temperature to 80° C., and the atomizer frequency to 30 Hz to obtain uniform fine powder.

### Example 9

Precisely weighed 25 g of sodium hyaluronate with molecular weight of 200 kDa, 15 g of acetylated sodium hyaluronate with molecular weight of 40 kDa, and 60 g of hydrolyzed sodium hyaluronate with molecular weight of 12 kDa. After mixing evenly, 3000 ml of purified water was added to make a 3.33% aqueous solution, which was then treated by spray-drying. Set the feeding temperature to 150° C., the discharging temperature to 80° C., and the atomizer frequency to 50 Hz to obtain uniform fine powder.

### Example 10

Precisely weighed 25 g of sodium hyaluronate with molecular weight of 180 kDa, 45 g of acetylated sodium hyaluronate with molecular weight of 50 kDa, and 30 g of hydrolyzed sodium hyaluronate with molecular weight of 15 kDa. After mixing evenly, 1000 ml of purified water was added to make a 10% aqueous solution, which was then treated by spray-drying. Set the feeding temperature to 120° C., the discharging temperature to 100° C., and the atomizer frequency to 30 Hz to obtain uniform fine powder.

### Example 11

Precisely weighed 60 g of sodium hyaluronate with molecular weight of 100 kDa, 10 g of acetylated sodium hyaluronate with molecular weight of 10 kDa, and 30 g of hydrolyzed sodium hyaluronate with molecular weight of 0.8 kDa. After mixing evenly, 1000 ml of purified water was added to make a 10% aqueous solution, which was then treated by spray-drying. Set the feeding temperature to 125° C., the discharging temperature to 90° C., and the atomizer frequency to 40 Hz to obtain uniform fine powder.

### Example 12

Precisely weighed 20 g of sodium hyaluronate with molecular weight of 500 kDa, 50 g of acetylated sodium hyaluronate with molecular weight of 80 kDa, and 30 g of hydrolyzed sodium hyaluronate with molecular weight of 20 kDa. After mixing evenly, 1000 ml of purified water was added to make a 10% aqueous solution, which was then treated by spray-drying. Set the feeding temperature to 125° C., the discharging temperature to 90° C., and the atomizer frequency to 40 Hz to obtain uniform fine powder.

### Example 13

Precisely weighed 10 g of sodium hyaluronate with molecular weight of 500 kDa, 60 g of acetylated sodium hyaluronate with molecular weight of 80 kDa, and 30 g of hydrolyzed sodium hyaluronate with molecular weight of 20 kDa. After mixing evenly, 2000 ml of purified water was added to make a 5% aqueous solution, which was then treated by spray-drying. Set the feeding temperature to 125° C., the discharging temperature to 90° C., and the atomizer frequency to 40 Hz to obtain uniform fine powder.

### Comparative Example 1

Precisely weighed 35 g of sodium hyaluronate with molecular weight of 280 kDa, 25 g of acetylated sodium hyaluronate with molecular weight of 25 kDa, 40 g of hydrolyzed sodium hyaluronate with molecular weight of 8 kDa, and the powders of the three materials were fully mixed under stirring with a stirrer, and stored for later use.

### Comparative Example 2

Precisely weighed 35 g of sodium hyaluronate with molecular weight of 280 kDa and 25 g of acetylated sodium hyaluronate with molecular weight of 25 kDa. After mixing evenly, 3000 ml of purified water was added to make a 3.33% aqueous solution, which was then treated by spray-drying. Set the feeding temperature to 125° C., the discharging temperature to 90° C., and the atomizer frequency to 40 Hz to obtain uniform fine powder.

### Comparative Example 3

Precisely weighed 35 g of sodium hyaluronate with molecular weight of 280 kDa and 40 g of hydrolyzed sodium hyaluronate with molecular weight of 8 kDa. After mixing evenly, 3000 ml of purified water was added to make a 3.33% aqueous solution, which was then treated by spray-drying. Set the feeding temperature to 125° C., the discharging temperature to 90° C., and the atomizer frequency to 40 Hz to obtain uniform fine powder.

### Comparative Example 4

Precisely weighed 25 g of acetylated sodium hyaluronate with molecular weight of 25 kDa and 40 g of hydrolyzed sodium hyaluronate with molecular weight of 8 kDa. After mixing evenly, 3000 ml of purified water was added to make a 3.33% aqueous solution, which was then treated by spray-drying. Set the feeding temperature to 125° C., the discharging temperature to 90° C., and the atomizer frequency to 40 Hz to obtain uniform fine powder.

TABLE 2
the amount of each component used in the examples

| | | | acetylated sodium | | hydrolyzed sodium | | |
|---|---|---|---|---|---|---|---|
| | sodium hyaluronate | | hyaluronate | | hyaluronate | | |
| | amount | molecular | amount | molecular | amount | molecular | Treatment |
| | (g) | weight(kDa) | (g) | weight(kDa) | (g) | weight(kDa) | mode |
| Example 1 | 20 | 400 | 10 | 100 | 70 | 18 | spray-drying |
| Example 2 | 30 | 250 | 30 | 30 | 40 | 10 | spray-drying |
| Example 3 | 35 | 280 | 25 | 25 | 40 | 8 | spray-drying |
| Example 4 | 25 | 300 | 20 | 30 | 55 | 5 | spray-drying |
| Example 5 | 40 | 250 | 35 | 28 | 45 | 3 | spray-drying |
| Example 6 | 30 | 210 | 20 | 20 | 50 | 3 | spray-drying |
| Example 7 | 50 | 150 | 15 | 45 | 35 | 3 | spray-drying |
| Example 8 | 55 | 300 | 15 | 10 | 30 | 15 | spray-drying |
| Example 9 | 25 | 200 | 15 | 40 | 60 | 12 | spray-drying |
| Example 10 | 25 | 180 | 45 | 50 | 30 | 15 | spray-drying |
| example11 | 60 | 100 | 10 | 10 | 30 | 0.8 | spray-drying |
| Example 12 | 20 | 500 | 50 | 80 | 30 | 20 | spray-drying |
| Example 13 | 10 | 500 | 60 | 80 | 30 | 20 | spray-drying |
| Comparative | 35 | 280 | 25 | 25 | 40 | 8 | Simply mixing |
| example 1 | | | | | | | |
| Comparative | 35 | 280 | 25 | 25 | / | / | spray-drying |
| example 2 | | | | | | | |
| Comparative | 35 | 280 | / | / | 40 | 8 | spray-drying |
| example 3 | | | | | | | |
| Comparative | / | / | 25 | 25 | 40 | 8 | spray-drying |
| example 4 | | | | | | | |

### Experimental Example 1: Solubility Property

Weighed 2.0 g of the powders obtained in Examples 1-13 and Comparative Examples 1-4 respectively into a 250 ml beaker, purified water at 25° C. was added and stirred at a stirring speed of 300 rpm, the time required for complete dissolution was recorded, and the results were shown in Table 3.

TABLE 3
the solubility properties of the compositions obtained in
Examples 1-13 and Comparative Examples 1-4

| | solubility property (min) |
|---|---|
| Example 1 | 18 |
| Example 2 | 14 |
| Example 3 | 12 |
| Example 4 | 11 |
| Example 5 | 13 |
| Example 6 | 12 |
| Example 7 | 17 |
| Example 8 | 15 |
| Example 9 | 16 |
| Example 10 | 16 |
| Example 11 | 21 |
| Example 12 | 20 |
| Example 13 | 25 |
| Comparative Example 1 | 60 |
| Comparative Example 2 | 38 |
| Comparative Example 3 | 29 |
| Comparative Example 4 | 46 |

As can be seen from table 3, the solubility property of the compositions obtained in Examples 1-13 was better, and the dissolution time was less than 25 min, while the dissolution time of the compositions obtained in Comparative Example 1-4 was longer, which was more than 29 min. The solubility property of the compositions of the present application was better.

Experimental Example 2: particle size detection Took about 10 g of the samples of Examples 1-13 and Comparative Examples 1-4 respectively, and recorded as W0, and placed them on a 200-mesh standard sieve to sieve, collected the samples that passed through the sieve, weighed, and recorded as W1, the passing rate=W1/W0×100%. And the results were shown in Table 4.

TABLE 4
the particle size detection results of the compositions obtained in
Examples 1-13 and Comparative Examples 1-4

| | Passing rate |
|---|---|
| Example 1 | 90% |
| Example 2 | 99% |
| Example 3 | 100% |
| Example 4 | 99% |
| Example 5 | 99% |
| Example 6 | 100% |
| Example 7 | 98% |
| Example 8 | 95% |
| Example 9 | 95% |
| Example 10 | 94% |
| Example 11 | 89% |
| Example 12 | 92% |
| Example 13 | 85% |
| Comparative Example 1 | 65% |
| Comparative Example 2 | 83% |
| Comparative Example 3 | 79% |
| Comparative Example 4 | 80% |

As can be seen from table 4, the composition obtained in Examples 1-13 passed through a 200-mesh sieve with a passing rate of over 85%, indicating that the particle size of the obtained composition was small and relatively uniform; while the passing rate of the comparative example was below 83%, indicating that the particle size of the composition of the present application was small and relatively uniform.

### Experimental Example 3: Absorbance Evaluation

Took 0.25 ml of the 2% aqueous solution prepared by the compositions obtained in Examples 1-13 and Comparative Examples 1-4 respectively, and smeared it on an area of 4 cm*4 cm on the inner skin of the volunteer's arm. During the smearing process, the volunteer recorded and evaluated the time required for complete absorption of the sample through subjective feelings, and the experimental results were shown in Table 5.

TABLE 5
absorption properties of the compositions obtained in
Examples 1-13 and Comparative Examples 1-4

| | Absorbance (s) |
|---|---|
| Example 1 | 21 |
| Example 2 | 16 |
| Example 3 | 15 |
| Example 4 | 12 |
| Example 5 | 13 |
| Example 6 | 11 |
| Example 7 | 18 |
| Example 8 | 17 |
| Example 9 | 19 |
| Example 10 | 19 |
| Example 11 | 23 |
| Example 12 | 21 |
| Example 13 | 24 |
| Comparative Example 1 | 40 |
| Comparative Example 2 | 28 |
| Comparative Example 3 | 31 |
| Comparative Example 4 | 26 |

As can be seen from table 5, the absorption rate of the compositions obtained in Examples 1-13 was faster, and the time required for complete absorption was below 24s: while the time required for complete absorption of the compositions described in Comparative Examples 1-4 was above 26s, indicating that the absorption rate of the compositions of the present application was fast.

### Application Example

### Transdermal Absorption Study:

To observe whether the hyaluronic acid composition with penetration-promoting effect can promote transdermal absorption of the target substance (tranexamic acid and astaxanthin), the following experiments were carried out.

### Application Verification Example 1

Formula containing tranexamic acid was listed below:

TABLE 6
formula composition table containing tranexamic acid

| | Validation formula | Control formula |
|---|---|---|
| | (percentage | (percentage |
| Raw material | content %) | content %) |
| caprylic decanoate triglyceride | 3 | 3 |
| hyaluronic acid composition | 1.0 | 0 |
| of Example 6 | | |
| butanediol | 4 | 4 |
| glycerin | 2 | 2 |
| tranexamic acid | 2 | 2 |
| carbomer | 0.15 | 0.15 |
| poloxamer | 0.4 | 0.4 |
| ethylparaben | 0.1 | 0.1 |
| triethanolamine | 0.07 | 0.07 |
| Purified water | To 100 | To 100 |

Experimental Steps:

1. Methodology of Tranexamic Acid Content

1.1 Establishment of HPLC Detection Method

1.1.1 Chromatographic Conditions

Chromatographic column: Hanbang C18 column (150 mm×4.6 mm, 5 μm)

Mobile phase: 0.23% sodium lauryi sulfate solution-methanol (60:40, V/V). Preparation of 0.23% sodium lauryl sulfate solution: took 18.3 g of sodium dihydrogen phosphate, added 800 ml of water to dissolve, added 8.3 ml of triethylamine and mixed evenly, then added 2.3 g of sodium lauryl sulfate, shook to dissolve, adjusted the pH value to 2.5 with phosphoric acid, added water to 100 ml, and shook well.

Flow rate: 0.8 ml/min

Column temperature: 35° C.

Detection wavelength: 220 nm

Injection volume: 20 μl

1.1.2 Linear Relationship Investigation

Precisely weighed 2.0059 g of the tranexamic acid as control substance, and diluted to 100 ml with mobile phase for later use. Took the appropriate amount of the stock solution respectively and diluted them with mobile phase to form a standard working solution with concentrations of 0.01, 0.01, 0.1, 1, 5, 10, and 20 mg/ml. Took 20 μL and measure the peak area (A) of each group at 220 nm through HPLC. The linear regression was performed on the concentration (ρ) with the A value and the standard curve was calculated.

Wherein, the results of the linear relationship investigation were shown in FIG. 1, with the peak area (A) as the ordinate and the mass concentration of tranexamic acid (μg/ml) as the abscissa.

It can be seen from FIG. 1 that the linear regression equation was y=429.57x−0.8027, r²=1, and the linearity between tranexamic acid and peak area was good at 1-20 mg/ml.

1.2 Evaluation of In Vitro Transdermal Absorption Performance

1.2.1 In Vitro Penetration Test

Took the abdominal skin of SD rats (the rats were purchased from the Qinglongshan Animal Breeding Farm in Jiangning District, Nanjing City, and the abdominal skin was peeled by the laboratory itself), after removing the fat, muscle, mucosa, and other tissues, sandwiched in the middle of Franz diffusion cell and placed in a 32° C. water bath. The supply pool of the control group was given 1 ml of

$${Q}_{n} = ( {VC}_{n} + {V}_{0} {\sum }_{i = 1}^{n - 1} {C}_{i} ) / A$$

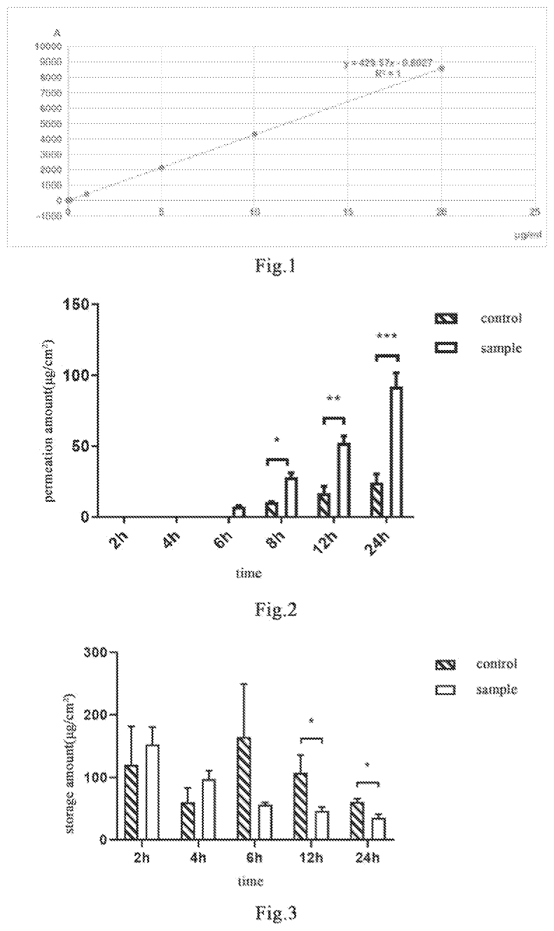

the control substance solution, and the supply pool of the sample group was given 1 ml of the same concentration of the verification formula. The receiving pool was filled with an appropriate amount of physiological saline and stirred at 400 r/min. At 0.5, 1.0, 2.0, 4.0, 6.0, 8.0, 12.0, and 24.0 hours, respectively, 5 ml of samples were taken from the receiving pool, and 5 ml of physiological saline was supplemented at the same time. After filtering the obtained sample solution through a 0.22 microporous filter membrane, detected according to the above chromatographic conditions described in 1.1.1, and substituted it into the regression equation to obtain the drug mass concentration. According to the mass concentration of the sample at each sampling point, the cumulative permeation amount per unit area (Qn) was calculated, the calculation formula was as follows, and the calculation results were shown in FIG. 2.

Cn and Ci were the drug mass concentration (μg/ml) measured at the nth and ith sampling points, respectively, V and V0 were respectively the receiving pool volume and sampling volume (ml), and A was the permeation area (cm²).

It can be seen from FIG. 2 that the cumulative permeation amount of tranexamic acid in the control formula for 24 hours was (24.3±14.9) μg/cm², and the cumulative permeation amount of tranexamic acid in the experimental formula for 24 hours was (92.2±23.3) μg/cm², which was about 4 times that of the control formula, and the difference between the groups was very significant (T test, P<0.01). Since the only difference between the two formulas was the hyaluronic acid composition, it shows that when the hyaluronic acid composition in Example 6 was added to the formulas as the transdermal delivery carrier, the transdermal absorption of transdermal acid can be significantly promoted.

1.2.2 In Vitro Retention Experiment

Took the abdominal skin of SD rats, after removing the fat, muscle, mucosa and other tissues, sandwiched in the middle of Franz diffusion cell and placed in a 32° C. water bath. The supply pool of the control group was given 1 ml of the control substance solution, and the supply pool of the sample group was given 1 ml of the same concentration of the verification formula. The receiving pool was filled with an appropriate amount of physiological saline and stirred at 400 r/min. Took out the skin tissue at 2.0, 4.0, 6.0, 12.0, and 24.0 hours respectively, washed off the residual drug on the skin surface, dried with filter paper, cut the effectively penetrated skin, added 5 ml of methanol-water (40:60), and pulverized it with a tissue grinder for 2 min, extracted with ultrasound for 30 min, took 2 ml of the supernatant and centrifuged at 12000 r/min for 10 min. After filtering the obtained sample solution through a 0.22 microporous filter membrane, detected according to the above chromatographic conditions described in 1.1, substituted it into the regression equation to obtain the drug mass concentration, and calculated the amount of storage. The results were shown in FIG. 3.

It can be seen from FIG. 3 that the skin storage amount of tranexamic acid in the verification formula group reached the highest value of 153.12 μg/cm² in 2 hours, which was higher than that of the control group, indicating that after adding the hyaluronic acid composition in Example 6, tranexamic acid can be quickly stored in the epidermis of the skin, facilitating subsequent transdermal absorption.

1.2.3 Experiment of Hydration Degree

Skin hydration refers to the ability of keratin in the outer layer of the skin or its degradation products to combine with water, and hydration degree refers to the water content of the stratum corneum. Hydration will increase the water content of the stratum corneum of the skin, thereby softening the tissue, filling the keratinocytes, increasing the water content in the intercellular space, and enlarging the interstitial space, which is conducive to the percutaneous penetration of active ingredients.

Volunteers first needed to wash the inner side of their forearms with clean water. After 30 minutes, pressed the moisture testing probe vertically on the surface of the skin to be tested, adjusted the pressing force according to the display on the instrument screen until the measurement result was displayed; then smeared the sample to be tested, measured the hydration degree again after 30 minutes, and calculated the increase rate of hydration degree according to the formula:

$$H = \frac{{H}_{1} - {H}_{0}}{{H}_{0}} \times 100 \% ;$$

Among which, H was the increase rate of hydration degree, H1 was the hydration degree measured after the sample was smeared, and H0 was the hydration degree measured before the sample was smeared.

The calculated increase rate of hydration degree was 78.61%.

### Application Verification Example 2: Research on In Vitro Permeation Test, In Vitro Retention Test and Hydration Degree Test of Other Examples and Comparative Examples

The hyaluronic acid compositions obtained from Examples 1-5, 7-13, and Comparative Examples 1-4 were tested according to the method described in Application Verification Example 1, and the cumulative penetration amount, skin storage amount, and hydration degree results were shown in Table 7.

TABLE 7
Results of cumulative penetration amount, skin storage amount and
hydration degree

| | Cumulative | Skin storage | |
|---|---|---|---|
| | penetration | amount | Increase rate |
| | amount | (μg/cm²) | of hydration |
| | (μg/cm²) (24 h) | (2 h) | degree (%) |
| Example 1 | 53.70 | 77.53 | 36.67 |
| Example 2 | 82.20 | 172.56 | 76.67 |
| Example 3 | 89.79 | 162.57 | 79.34 |
| Example 4 | 95.09 | 147.17 | 64.52 |
| Example 5 | 84.54 | 140.41 | 74.07 |
| Example 7 | 78.82 | 110.47 | 65.38 |
| Example 8 | 65.74 | 93.76 | 51.13 |
| Example 9 | 61.10 | 109.96 | 58.72 |
| Example 10 | 60.46 | 84.06 | 41.87 |
| Example 11 | 49.64 | 72.62 | 33.33 |
| Example 12 | 56.64 | 74.25 | 36.36 |
| Example 13 | 35.67 | 55.32 | 31.87 |
| Comparative Example 1 | 32.81 | 64.66 | 18.09 |
| Comparative Example 2 | 29.05 | 56.17 | 28.12 |
| Comparative Example 3 | 14.75 | 53.76 | 23.10 |
| Comparative Example 4 | 13.87 | 47.04 | 19.28 |
| Control formula | 24.3 | 51.60 | 30.10 |

It can be seen from Table 7 that in the verification formula prepared using the hyaluronic acid compositions described in Examples 1-5 and 7-13, the cumulative penetration amount and skin storage amount were very high, and the increase rate of the hydration degree was also very high, this was because the hyaluronic acid or a salt thereof in the hyaluronic acid composition of the examples had a good moisturizing effect, and the acetylated hyaluronic acid or a salt thereof can also promote skin hydration, thereby softening the tissues, filling the keratinocytes, increasing the water content in the intercellular space, opening the skin barrier, and free nutrients as well as nutrients combined with the hydrolyzed hyaluronic acid or a salt thereof and hyaluronic acid or a salt thereof entered the skin, thereby promoting active absorption. This indicated that the hyaluronic acid composition described in the examples can promote the rapid penetration of tranexamic acid through the skin and store it in the epidermis, which was beneficial to subsequent transdermal absorption, thus illustrating the hyaluronic acid composition of the present application can promote the absorption of active ingredients.

### Application Verification Example 3

Formulas containing astaxanthin were listed below.

TABLE 8
Formulas containing astaxanthin

| | Verification formula | Control formula |
|---|---|---|
| | (content %) | (content %) |
| caprylic decanoate triglyceride | 3 | 3 |
| astaxanthin | 0.1 | 0.1 |
| hyaluronic acid composition | 0.4 | 0 |
| of Example 6 | | |
| Hydrogenated lecithin | 0.5 | 0.5 |
| Xanthan gum | 0.1 | 0.1 |
| butanediol | 4 | 4 |
| glycerin | 2 | 2 |
| carbomer | 0.1 | 0.1 |
| triethanolamine | 0.07 | 0.07 |
| Purified water | To 100 | To 100 |

1. Materials and Methods

1.1 Establishment of HPLC Detection Method

1.1.1 Chromatographic Conditions

Chromatographic column: Hanbang C18 column (150 mm×4.6 mm, 5 μm)

Mobile phase: methanol-water (95:5, VN)

Flow rate: 1.0 ml/min

Column temperature: 25° C.

Detection wavelength: 475 nm

Injection volume: 20 μL

1.1.2 Drawing of Standard Curve

Precisely weighed 5.0 mg of astaxanthin as control substance, added 1 ml of dichloromethane, then diluted to 50 ml with methanol to constant volume, set aside, and stored at −4° C. in the dark. Took an appropriate amount of stock solution, diluted with methanol to form standard working solutions with concentrations of 0.5, 1.0, 2.0, 5.0, 10.0, and 20.0 μg/mil, respectively, and prepared them for immediate use. Took 20 μL and measured the peak area (A) of each group at 475 nm through HPLC. The linear regression was performed on the concentration (p) with the A value and the standard curve was calculated.

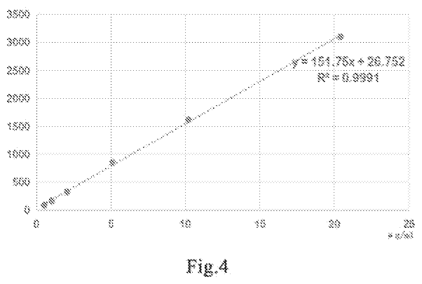

Among which, the results of the linear relationship investigation were shown in FIG. 4, with the peak area (A) as the ordinate and the mass concentration of astaxanthin (μg/ml) as the abscissa.

It can be seen from FIG. 4 that the linear regression equation was y=151.75x+26.752, r²=0.9991, and the astaxanthin had a good linearity with the peak area at 0.5-20 μg/ml.

1.2 In Vitro Transdermal Absorption Performance Evaluation

1.2.1 Preparation of Control Solution for In Vitro Evaluation

Dissolved 1 g of hydroxyethyl cellulose (HEC) in 80 ml of water, let it stand for swelling, stirred evenly, and set aside. Dispersed 0.1200 g of astaxanthin oil and 6.0 mg of astaxanthin standard product in 5 g of glycerol, respectively, after uniform dispersion, added to HEC gel, and then diluted to 100 ml with water.

1.2.2 In Vitro Retention Experiment

Took the abdominal skin of SD rats, after removing the fat, muscle, mucosa and other tissues, sandwiched in the middle of Franz diffusion cell and placed in a 32° C. water bath. The supply pool of the control group was given 1 ml of two control substance solutions respectively, the supply pool of the sample group was given 1 ml of the same concentration of samples, and the receiving pool was filled with an appropriate amount of 1% Tween 80 solution, and stirred. The tissues were taken out at 2.0 h, 4.0 h, 6.0 h, 12.0 h, and 24.0 h, respectively, and the surface residue was washed 5 times with physiological saline, dried with filter paper, cut the effectively penetrate skin, added 4 ml of solvent (methanol:dichloromethane=3:1), and pulverize with a tissue grinder for 2 minutes, extracted with ultrasound for 30 minutes, added 1 ml of 1% KOH methanol solution, shook well, reacted at 30° C. for 30 minutes, centrifuged at 5000 r/min for 5 minutes at 4° C., and filtered with a 0.22 μm filter membrane. The average value of the obtained data was shown in Table 9.

TABLE 9
Skin storage amount results

| | | | | | |
|---|---|---|---|---|---|
| time/h | 2 | 4 | 6 | 12 | 24 |
| Storage amount of | 0.026 | 0.023 | 0.026 | 0.026 | 0.038 |
| submuscular fluid/μg/cm² | | | | | |
| Storage amount of the | — | — | — | — | 0.011 |
| control group/μg/cm² | | | | | |

It can be seen from Table 9 that the 24 h full face storage amount of the control formula group and the verification formula group were 0.011 μg/cm² and 0.038 μg/cm² respectively, and there was a significant difference in the storage amount between the two groups. The only difference between the two groups was that the content of the hyaluronic acid composition was different (the verification formula contained 0.4% hyaluronic acid composition, and the control formula did not contain hyaluronic acid), therefore it was considered that the hyaluronic acid composition of Example 6 played a key role in the transdermal absorption of astaxanthin.

1.2.2 Hydration Degree Experiment

Skin hydration refers to the ability of keratin in the outer layer of the skin or its degradation products to combine with water, and hydration degree refers to the water content of the stratum corneum. Hydration will increase the water content of the stratum corneum of the skin, thereby softening the tissues, filling the keratinocytes, increasing the water content in the intercellular spaces, and enlarging the interstitial space, which is conducive to the percutaneous penetration of active ingredients.

Volunteers first needed to wash the inner side of their forearms with clean water, and after 30 minutes, pressed the moisture testing probe vertically on the surface of the skin to be tested, adjusted the pressing force according to the display on the instrument screen until the measurement result was displayed, then smeared the sample to be tested, measured the hydration degree again after 30 minutes, and calculated the increase rate of hydration degree according to the formula:

$$H = \frac{{H}_{1} - {H}_{0}}{{H}_{0}} \times 100 \% ;$$

among which. H was the increase rate of hydration degree, H1 was the hydration degree measured after the sample was smeared, and H0 was the hydration degree measured before the sample was smeared.

The resulting increase rate of hydration degree was 86.44%.

### Application Verification Example 4: Research on In Vitro Retention Experiments and Hydration Degree Experiments of Other Examples and Comparative Examples

The hyaluronic acid compositions obtained in Examples 1-5, 7-13 and Comparative Examples 1-4 were tested according to the method described in Application Verification Example 3, and the results of the skin storage amount and hydration degree were shown in Table 10.

TABLE 10
Results of skin storage amount and hydration degree

| | skin storage amount | Increase rate of |
|---|---|---|
| | (μg/cm²) (24 h) | hydration degree (%) |
| Example 1 | 0.028 | 32.87 |
| Example 2 | 0.051 | 90.33 |
| Example 3 | 0.049 | 89.34 |
| Example 4 | 0.058 | 90.37 |
| Example 5 | 0.042 | 88.47 |
| Example 7 | 0.034 | 65.22 |
| Example 8 | 0.032 | 68.18 |
| Example 9 | 0.033 | 59.09 |
| Example 10 | 0.034 | 46.67 |
| Example 11 | 0.025 | 38.62 |
| Example 12 | 0.023 | 31.10 |
| Example 13 | 0.018 | 30.03 |
| Comparative Example 1 | 0.012 | 27.14 |
| Comparative Example 2 | 0.016 | 23.33 |
| Comparative Example 3 | 0.010 | 18.38 |
| Comparative Example 4 | 0.012 | 18.75 |
| Control formula | 0.011 | 35.25 |

As can be seen from Table 10, there was a significant difference in the skin storage amount within 24 hours between the hyaluronic acid compositions described in the Examples and the Comparative Examples, indicating that the hyaluronic acid compositions described in the Examples can promote the transdermal absorption of astaxanthin; in addition, there was also a significant difference in the increase rate of hydration degree compared to that of the Comparative Examples. Since hydration degree represents the amount of water in the stratum corneum, hydration can increase the water content in the stratum corneum of the skin, thereby softening the tissues, filling the keratinocytes, increasing the water content in the intercellular spaces, and enlarging the interstitial space, which was conducive to the percutaneous penetration of active ingredients, which further showed that the hyaluronic acid composition described in the present application can promote the absorption of active ingredients.

In summary, the present application employs a hyaluronic acid composition, a hyaluronic acid or a salt thereof is equivalent to a very small network structure, which is distributed on the surface of the skin; an acetylated hyaluronic acid or a salt thereof has acetyl and is lipophilic, thus it can be skin friendly with the skin and form channels: a hydrolyzed hyaluronic acid or a salt thereof can quickly penetrate into the skin layer through the channels formed by the acetylated hyaluronic acid or a salt thereof, the hydrolyzed hyaluronic acid or a salt thereof can quickly open the skin barrier by mixing with the hyaluronic acid or a salt thereof, and free nutrients, the hydrolyzed hyaluronic acid or a salt thereof as well as nutrients combined with the hyaluronic acid or a salt thereof enter the skin, thereby promoting the absorption of active molecules. In addition, the hyaluronic acid composition can improve the hydration degree, thereby softening the tissues, filling the keratinocytes, increasing the water content in the intercellular space, and enlarging the interstitial space, which is conducive to the percutaneous penetration of active ingredients.

The above are only preferred examples of the present application, and are not intended to limit the present application to other forms. Any skilled person in the art may use the technical content disclosed above to change or modify it as an equivalent example with the equivalent changes. However, any simple modifications, equivalent changes and modifications made to the above examples according to the technical essence of the present application without departing from the content of the technical solutions of the present application still belong to the protection scope of the technical solution of the present application.

1-22. (canceled)

23. A hyaluronic acid composition, comprising:
a hyaluronic acid or a salt thereof;
an acetylated hyaluronic acid or a salt thereof; and
a hydrolyzed hyaluronic acid or a salt thereof.

24. The hyaluronic acid composition according to claim 23, wherein
the hyaluronic acid composition consists of a hyaluronic acid or a salt thereof, an acetylated hyaluronic acid or a salt thereof, and a hydrolyzed hyaluronic acid or a salt thereof.

25. The hyaluronic acid composition according to claim 23, wherein based on the weight percentage in the hyaluronic acid composition,
the hyaluronic acid or a salt thereof is 20-60%, preferably 25-55%, more preferably 25-40%;
the acetylated hyaluronic acid or a salt thereof is 10-50%, preferably 15-45%, more preferably 20-35%, more preferably 20-30 wt %; and
the hydrolyzed hyaluronic acid or a salt thereof is 30-70%, preferably 30-60%, more preferably 40-55%.

26. The hyaluronic acid composition according to claim 23, wherein
the molecular weight of the hyaluronic acid or a salt thereof is 100 k-500 kDa, preferably 150 k-300 kDa, more preferably 210 k-300 kDa;
the molecular weight of the acetylated hyaluronic acid or a salt thereof is 10 k-100 kDa, preferably 10 k-50 kDa, more preferably 20 k-30 kDa; and
the molecular weight of the hydrolyzed hyaluronic acid or a salt thereof is 0.8 k-20 kDa, preferably 3 k-15 kDa, more preferably 3 k-10 kDa.

27. A method for preparing a hyaluronic acid composition, comprising the following steps:
dissolving a hyaluronic acid or a salt thereof, an acetylated hyaluronic acid or a salt thereof, and a hydrolyzed hyaluronic acid or a salt thereof to obtain a solution;
spray-drying the resulting solution to obtain a hyaluronic acid composition.

28. The method according to claim 27, wherein based on the mass percentage in the solution, the sum of the hyaluronic acid or a salt thereof, acetylated hyaluronic acid or a salt thereof and hydrolyzed hyaluronic acid or a salt thereof is 1-10%.

29. The method according to claim 27, wherein the feeding temperature of spray-drying is 120-150° C., the discharging temperature is 80-100° C., preferably, the atomization frequency is 30-50 Hz.

30. The method according to claim 28, wherein based on the weight percentage in the hyaluronic acid composition,
the hyaluronic acid or a salt thereof is 20-60%, preferably 25-55%, more preferably 25-40%;
the acetylated hyaluronic acid or a salt thereof is 10-50%, preferably 15-45%, more preferably 20-35%, more preferably 20-30 wt %; and
the hydrolyzed hyaluronic acid or a salt thereof is 30-70%, preferably 30-60%, more preferably 40-55%.

31. The method according to claim 28, wherein
the molecular weight of the hyaluronic acid or a salt thereof is 100 k-500 kDa, preferably 150 k-300 kDa, more preferably 210 k-300 kDa;
the molecular weight of the acetylated hyaluronic acid or a salt thereof is 10 k-100 kDa, preferably 10 k-50 kDa, more preferably 20 k-30 kDa; and
the molecular weight of the hydrolyzed hyaluronic acid or a salt thereof is 0.8 k-20 kDa, preferably 3 k-15 kDa, more preferably 3 k-10 kDa.

32. A composition for promoting the absorption of an active ingredient, wherein the composition comprises an active ingredient and the hyaluronic acid composition according to claim 23 or a hyaluronic acid composition prepared by a preparation method comprising the following steps:
dissolving a hyaluronic acid or a salt thereof, an acetylated hyaluronic acid or a salt thereof, and a hydrolyzed hyaluronic acid or a salt thereof to obtain a solution;
spray-drying the resulting solution to obtain the hyaluronic acid composition.

33. The composition according to claim 32, wherein the active ingredient is a water-soluble active ingredient and/or an oil-soluble active ingredient; preferably, the water-soluble active ingredient is tranexamic acid, nicotinamide, vitamin C, ergothioneine, small molecule peptides containing 2-10 amino acids, aminobutyric acid, deoxyribonucleic acid, pro-xylane, or ectoine; and the oil-soluble active ingredient is astaxanthin, salicylic acid, ferulic acid, phenylethyl resorcinol, resveratrol, undecylenoyl phenylalanine, or ethyl bis(iminomethyl)guaiacol manganese chloride.

34. The composition according to claim 32, wherein the mass ratio of the active ingredient to the hyaluronic acid composition is 1:5 to 5:1, preferably 1:4 to 4:1.

35. Use of the hyaluronic acid composition according to claim 23 or a hyaluronic acid composition prepared by a preparation method, in improving the absorption of an active ingredient, preferably, the active ingredient is a water-soluble active ingredient and/or an oil-soluble active ingredient, wherein
the preparation method comprises the following steps:
dissolving a hyaluronic acid or a salt thereof, an acetylated hyaluronic acid or a salt thereof, and a hydrolyzed hyaluronic acid or a salt thereof to obtain a solution;
spray-drying the resulting solution to obtain the hyaluronic acid composition.

36. Use according to claim 35, wherein
the water-soluble active ingredient is tranexamic acid, nicotinamide, vitamin C, ergothioneine, small molecule peptides containing 2-10 amino acids, aminobutyric acid, deoxyribonucleic acid, pro-xylane, or ectoine; and the oil-soluble active ingredient is astaxanthin, salicylic acid, ferulic acid, phenylethyl resorcinol, resveratrol, undecylenoyl phenylalanine, or ethyl bis(iminomethyl)guaiacol manganese chloride.

37. A product, comprising the composition according to claim 32.

38. The product according to claim 37, wherein based on the mass percentage in the product, the hyaluronic acid composition is 0.1-2%, preferably 0.5-1.5%.

39. The product according to claim 37, wherein the product is a skin care product, a disinfection product, a medicine or a dressing, a gel medical device.

40. The product according to claim 39, wherein the skin care product is toner, essence, cream, mask or lotion.

41. Use of the hyaluronic acid composition according to claim 23 or a hyaluronic acid composition prepared by a preparation method, in the field of products, preferably, the hyaluronic acid composition is 0.1-2%, preferably 0.5-1.5%, based on the weight percentage in the product, wherein
the preparation method comprises the following steps:
dissolving a hyaluronic acid or a salt thereof, an acetylated hyaluronic acid or a salt thereof, and a hydrolyzed hyaluronic acid or a salt thereof to obtain a solution;
spray-drying the resulting solution to obtain the hyaluronic acid composition.

42. The use according to claim 41, wherein the product is a skin care product, a disinfection product, a medicine or a dressing, or a gel medical device.
